(19) United States
(12) Patent Application Publication
(10) Pub. No.: US 2003/0114529 A1
(43) Pub. Date: Jun. 19, 2003

(54) Hydroxamic acid thrombospondin peptide analog that inhibits aggrecanase activity

(76) Inventors: Michael Tortorella, Chicago, IL (US); Jinhai Wang, Foster City, CA (US); Rodney L. Balhorn, Livermore, CA (US)

Correspondence Address: HOWARD M. PETERS, PETERS, VERNY, JONES & SCHMITT, L.L.P., Suite 6, 385 Sherman Avenue, Palo Alto, CA, 94306, US

(21) Appl. No.: 10/192,283

(22) Filed: Jul. 9, 2002

Related U.S. Application Data
(60) Provisional application No. 60/303,989, filed on Jul. 9, 2001.

(51) Int. Cl. A61K 31/19

(52) U.S. Cl. 514/575

(57) ABSTRACT

The present invention concerns the generation of hydroxamic acid thrombospondin-peptide analogs that inhibit aggrecanase activity. These analogs are useful in the treatment of diseases characterized by cartilage degradation, such as osteoarthritis, rheumatoid arthritis spondylarthropathies, and septic arthritis. The invention describes a novel small molecule, enzyme inhibitor that binds both the enzyme and its naturally occurring substrate.

### RELATED APPLICATIONS

This application is a continuation-in-part of U.S. Ser. No. 60/303,989, filed Jul. 9, 2001 which is incorporated herein by reference in its entirety.

### BACKGROUND OF THE INVENTION

1. Field of the Invention

The present invention concerns peptide analog inhibitors for aggrecanase which essentially stop the in vivo action of aggrecanase. These inhibitors are useful in the treatment of a variety of human disease conditions including diseases such as osteoarthritis and rheumatoid arthritis.

2. Description of Related Art

Aggrecanase 1 is one of two novel cartilage-degrading metalloproteases purified from bovine nasal cartilage cultures stimulated with interleukin-1[1-2]. The enzyme shares 40-50% sequence homology with aggrecanase 2 (ADAMTS-11/5), ADAMTS-1 (METH-1) and ADAMTS-8 (METH-8) as well as lower degrees of homology with other members of the a disintegrin and metalloprotease with a thrombospondin motif (ADAMTS) family (3-7). All members of the ADAMTS family consist of an amino-terminal propeptide domain, a metalloproteinase domain that requires zinc for enzymatic activity, and a disintegrin-like domain, resembling the structural elements of the reprolysin family of metalloproteases that includes the a disintegrin and metalloprotease (ADAM) and snake venom metalloproteases (8). Unlike typical ADAM proteins that are membrane-anchored and have a transmembrane domain and cytoplasmic domain in the carboxyl terminal region, the C-terminus of ADAMTS proteins contains a varying number of thrombospondin type-1 (TSP-1) motifs, the sequence of which is the conserved motif in thrombospondin 1 and 2 (8). The sequence GGWGPWGPWG (Seq. No. 3) within the TSP-1 motif of ADAMTS-4 binds to the glycosaminoglycans (GAG) of aggrecan. This binding of aggrecanase to aggrecan through the TSP-1 motif is necessary for enzymatic cleavage of aggrecan (9).

Aggrecan is a large chondroitin sulfate proteoglycan that accounts for about 10% of the dry weight of cartilage [10,11]. It consists of three globular domains, G1, through which it interacts with hyaluronan (HA), G2, and G3 at the C-terminus of the molecule. The core protein between G2 and G3 is highly substituted with the glycosaminoglycans (GAG) keratan sulfate and chondroitin sulfate chains. Aggrecan is usually found as part of a large aggregate with HA containing approximately 100 proteoglycan molecules per HA molecule. The molecule carries a large number of fixed negatively charged groups on the GAGs that results in high osmotic pressure in the tissue, thus allowing aggrecan to swell and hydrate the framework of collagen fibrils in cartilage, providing the tissue its properties of compressibility and resilience. The interglobular domain (IGD) of aggrecan between the G1 and G2 domain has been shown to be susceptible to proteolytic cleavage by ADAMTS-4/ADAMTS-5 between residues Glu³⁷³-Ala³⁷⁴, but not at the MMP site between residues Asn³⁴¹-Phe³⁴²(12). In addition, it has recently been demonstrated that human recombinant ADAM-TS4 and ADAM-TS5 both cleave aggrecan preferentialy at four sites located in the chondroitin sulfate-rich region between G2 and G3 at the Glu¹⁴⁸⁰-Gly ¹⁴⁸¹, Glu¹⁶⁶⁷-Gly¹⁶⁶⁸, Glu¹⁷⁷¹-Ala¹⁷⁷² and Glu¹⁸⁷¹-Leu¹⁸⁷² bonds (12). Loss of aggrecan leads to cartilage dysfunction typically seen in diseases such as osteoarthritis and rheumatoid arthritis. Therefore, blocking aggrecanase cleavage of aggrecan may prove to be useful in treating patients who suffer from arthritic diseases.

In this patent, we describe the use of a novel small molecule hydroxamic acid thrombospondin peptide analog that inhibits ADAMTS-4/ADAM-TS5, and prevents aggrecanase-1 from binding and cleaving native aggrecan. This novel inhibitor may prove useful in treating diseases characterized by cartilage breakdown.

References of specific and general interest include:

1. M. D. Tortorella, et al. Purification and cloning of aggrecanase-1: a member of the ADAMTS family of proteins. Science 1999; 284:1664-6.

2. I. Abbaszade, et al. Cloning and characterization of ADAMTS11, an aggrecanase from the ADAMTS family. J. Biol Chem 1999; 274:23443-50.

3. K. Kuno, et al. (1997) Genomics 46, 466-71.

4. A. Colige, et al. (1997) Proc. Natl. Acad. Sci. U.S.A. 94, 2374-2379.

5. B. L. Tang, et al. (1999) FEBS Lett. 445, 223.5.

6. F. Vasquez, et al. (1999) J. Biol Chem 274, 23349-23357.

7. T. L. Hurskainen, et al. (1999) J. Biol Chem 274, 25555-25563.

8. G. P. Kaushal, et al. The new kids on the block: ADAMTSs, potentially multifunctional metalloproteinases of the ADAM family. J. Clin Invest 2000; 105:1335-7.

9. M. Tortorella, et al. The thrombospondin motif of aggrecanase-1 (ADAMTS-4) is critical for aggrecan substrate recognition and cleavage. J. Biol Chem 2000; 275(33): 25791-1.

10. M. Paulsson, et al. Extended and globular protein domains in cartilage proteoglycans. Biochem J. 1987; 245: 763-72.

11. T. E. Hardingham, et al. Aggrecan, the chondroitin sulfate/keratan sulfate proteoglycan from cartilage. In Articular Cartilage and Osteoarthritis (Kuettner, K. E., Schleyerbach, R. Peyton, J. G., and Hascall, V. C.) eds. 1992; Raven Press, New York: 5-20.

12. M. D. Tortorella, et al. Sites of aggrecan cleavage by recombinant human aggreanase-1 (ADAMTS-4). J. Biol Chem 2000; 275: 18566-73.

13. R. W. Farndale, et al. Improved quantitation and discrimination of sulphated glycosaminoglycans by use of dimethylmethylene blue. Biochim Biophys Acta 1986; 883: 173-7.

14. E. C. Arner, et al. Cytokine-induced cartilage proteoglycan degradation is mediated by aggrecanase. Osteoarthritis and Cartilage 1998; 6:214-28.

The above discussion demonstrate that a need exists for therapeutic agents and methods to treat osteoarthritis and rheumatoid arthritis. The present application provides solutions.

15. N. D. Rawlings, et al. (1995) Methods Enzymol. 248, 183-228.

16. T. G. Wolsberg, et al. (1996) Dev. Biol. 180, 389-340.

17. P. Bornstein, et al. (1994) Methods Enzymol. 245, 62-85.

18. V. C. Hascall, et al. (1969) J. Biol. Chem. 244, 2384-2396

U.S. Patents of interest include: U.S. Pat. No. 6,057,336; U.S. Pat. No. 6,180,334; and U.S. Pat. No. 5,872,209.

World or foreign patents include: EP 1081137; EP 1041702; WO 2000059874; WO 2000059285; WO 2000075108; WO 200009485; WO 2000009492; WO 9965867; WO 9964406; WO 9951572; WO 9909000; WO 9905291; WO 9851665; WO 9740072; WO 9731931; WO 9718207; and WO 9633166.

All articles, references, patents, applications, standards and the like cited herein by reference in their entirety.

### SUMMARY OF THE INVENTION

This invention relates to the use of hydroxamic acid thrombospondin peptide analog compounds for the treatment of diseases characterized by cartilage degradation including osteoarthritis, rheumatoid arthritis, spordylarthropathies, septic arthritis and the like. It also includes the use of these analog compounds for the inhibition of ADAMTS-4/ADAMTS-5 in vivo and in vitro.

These novel peptide analogs involve structures comprising hydroxamic acid derivatives. The analog design enables enhanced potency and selectivity by providing multiple binding and inhibitory activities in a single structure. The hydroxamic acid derivatives inhibit ADAMTS4/ADAMTS5 through chelation with zinc in the catalytic domain, whereas, analogues of the peptide sequence GGWGPWGPWP (Seq. No. 3) inhibit enzyme activity through binding to the glycosaminoglycan chains of the aggrecan substrate.

Specific hydroxamic acid analogs include, but are not limited to the following:

| | | | |
|---|---|---|---|
| H—O—NH—(C═O)—CH₂—CH(CH₂ CH(CH₃)₂)—(C═O)—V- | | | |
| ″ -SQAGGWGPWGPWGDSSAT; | (˜11′A) | AS323 | (Seq. No.9) |
| ″ -SNISQAGGWGPWGPWGDSSAT; | (˜22′A) | AS324 | (Seq. No.10) |
| ″ -LQDSNISQAGGWGPWGPWGDSSAT; | (˜33′A) | AS325 | (Seq. No.11) |
| ″ -MDQLQDSNISQAGGWGPWGPWGDSSAT. | (˜44′A) | | (Seq. No.12) |

The valine amino acid in the above structure an be replaced with any amino acid, e.g, phe, ala, tyr. etc. Preferred amino acids that can be substituted for valine are shown in FIGS. 1A to 1J.

The present invention also concerns a method of therapy for osteoarthritis and rheumatoid arthritis by administering a therapeutically effective amount of the peptide to a manmal, preferably a human being.

### BRIEF DESCRIPTION OF THE FIGURES

The following describe the structures of the hydroxamic acid analogues.

FIG. 1A is JWC-95.

FIG. 1B is JWD-52.

FIG. 1C is JWD-97.

FIG. 1D is JWD-48.

FIG. 1E is JWD-40.

FIG. 1F is JWD-39.

FIG. 1G is JWD-100.

FIG. 1H is XN908.

FIG. 1I is XS309.

FIG. 1J is JWC-96.

FIG. 2 demonstrates the protective effect of JSD40 against aggrecan degradation in human osteoarthritic cartilage.

FIG. 3 is a schematic representation which demonstrates the protective effect of the peptide, GGWGPWGPWGDCSRTCGGG (Sequence No. 14), against degradation of aggrecan by aggrecanase. Lane 1; intact aggrecan. Lane 2; aggrecan and aggrecanase. Lane 3; aggrecan, aggrecanase and peptide.

FIG. 4 describes the structures of the thrombospondin peptide analogues that bind to the glycosaminoglycan chains of aggrecan.

FIG. 5 is a schematic representation of an example of a hydroxamic acid thrombospondin peptide inhibitor of aggrecanase.

FIG. 6 is a schematic representation of the inhibition of ADAMTS4 (aggrecanase 1) by the hydroxamic acid thombospondin peptide analog compounds.

### DETAILED DESCRIPTION OF THE INVENTION AND PREFERRED EMBODIMENTS

Definitions

As used herein:

“Amino acids” refer to natural and synthetic amino acids. The standard designations A, D, S, etc. and gly, ala, cys, etc. are used to identify the natural amino acids.

The definitions for chemicals reagents and the like are conventional in the art.

The hydroxamic acid thrombospondin peptide analog provides a novel system for specific delivery of the hydroxamic acid to the site of interaction between the enzyme and its substrate.

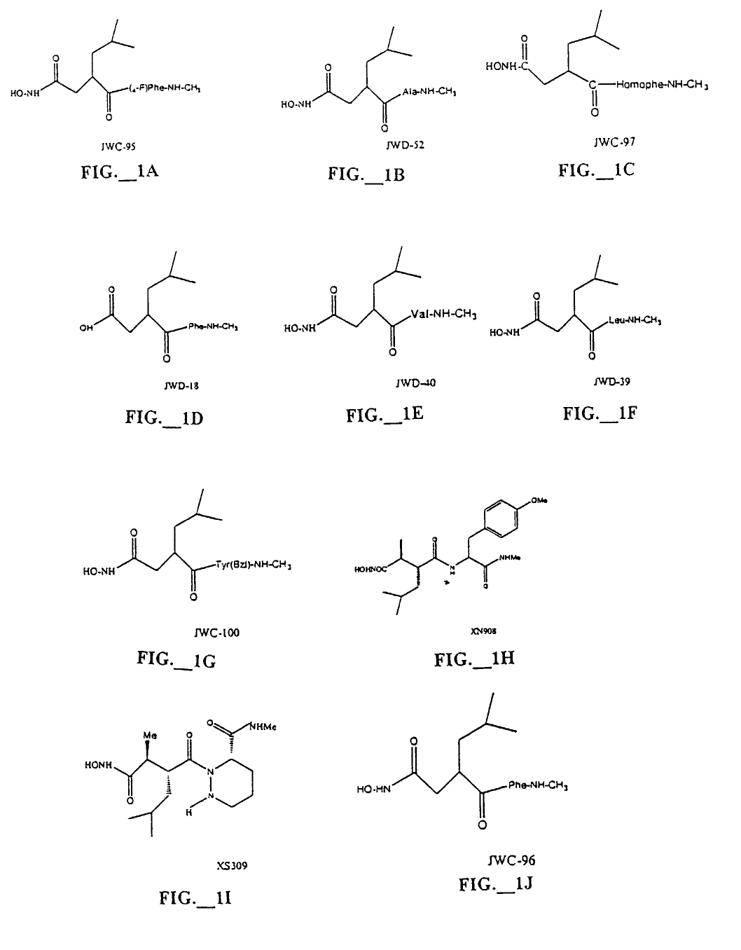

A series of newly synthesized hydroxamic acids (FIG. 1) were tested for their ability to inhibit recombinant ADAMTS-4/ADAM-TS5 as well a several matrix metalloproteinases (MMPs). In this study aggrecan, at a concentration of 500 nM, was incubated for 2 hours at 37° C. with 5 nM of either ADAMTS-4/ADAM-TS5 in the presence or absence of one of the hydroxamic acids at a concentration range from 0.1 to 1000 nM. Subsequently, the fractions were analysed for cleavage of aggrecan at the Glu¹⁴⁸⁰-¹⁴⁸¹Gly site by Western Blot analysis, using a specific antibody recognizing the new C-terminus GELE¹⁴⁸⁰. Inhibition of cleavage was determined by scanning densitometry and the Ki values were determined (Table 1). JWD40 displayed the greatest potency against aggrecanae with Ki vaue of 17 nM. The same compound showed a log less potency against MMP-3, and twofold less potent against MMP-1 and MMP-2.

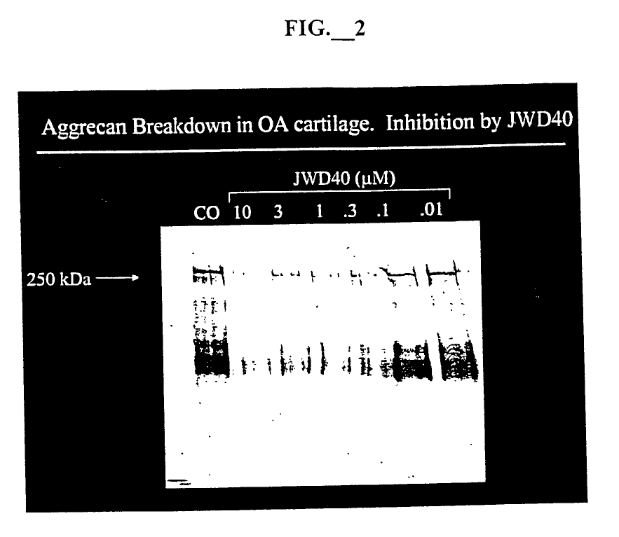

The same compounds were tested for the ability to block aggrecan cleavage in an in vitro model of cartilage degradation. Pig articular cartilage explants were cultured for 72 hours in the presence of IL-α, with or without one of the inhibitors at a concentration range og 0.1 to 10 μM. Culture media were assessed for the level of glycosaminoglycan chains (GAG) by dimethyl methylene blue (DMMB) assay, as a measure of aggrecan breakdown and IC50 values were calculated (Table 1). JWD40 was found to be very effective in protecting against IL-1 induced aggrecan degradation. Therefore, we tested JWD40 for its ability to prevent aggrecan degradation in osteoarthritic (OA) cartilage. Human OA cartilage explants were cultured with or without JWD40, 0.1 to 10 μM. After 48 hours, media were analyzed for the presence of aggrecan products generated by cleavage by aggrecanase, containing the new N-terminus ³⁷⁴ARGSV. The compound blocked the release of this fragment in a concentration-dependent manner (FIG. 2).

An aggrecan binding peptide sequence derived from thrombospondin motif of aggrecanase-1 was tested for its ability to protect aggrecan from degradation by aggrecanase. Full length aggrecanase-1 (ADAMTS4), 5 nM, was incubated with native aggrecan, 500 nM, for 5 hours at 37° C. in the presence of the thrombospondin peptide GGWGPWGPWGDCSRTCGGG, 100 μM (Seq. No. 14). Aggrecan cleavage products were measured by Western blot analysis using the monoclonal antibody MAB2035.

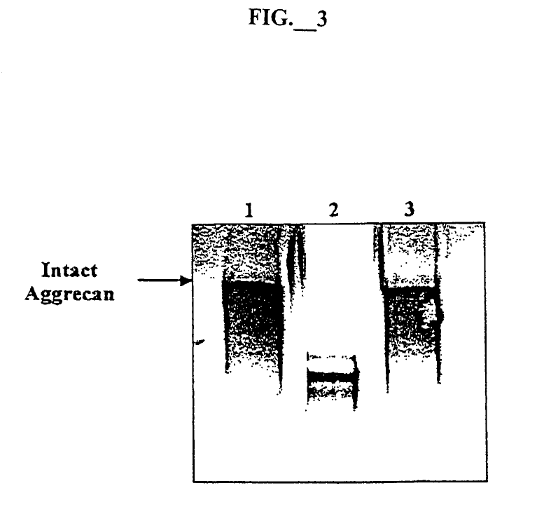

Aggrecanase activity was demonstrated by the reduction or disappearance of high molecular weight aggrecan and the appearance of lower molecular weight fragments. Neither the loss of high molecular weight aggrecan nor the appearane of aggrecan fragments could be demonstrated in cultures containing the thrombospondin motif peptide. Thus the peptide protects aggrecan from cleavage by aggrecanase by preventing aggrecanase from binding to aggrecan (FIG. 3).

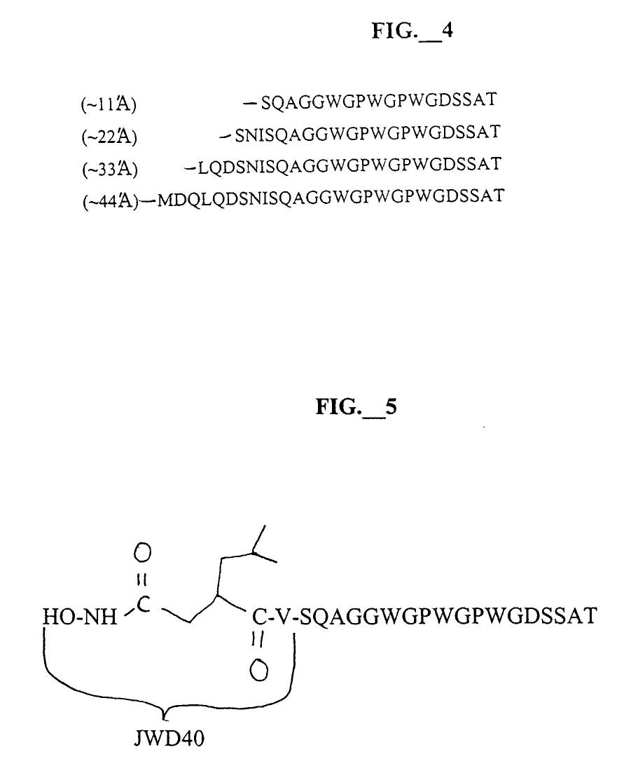

The novel peptide analogues are produced by conjugating a hydroxamic acid derivative such as JWD40 to the N-terminus of the novel aggrecan binding peptides described in FIG. 4.

JWD40 is a potent and semiselective inhibitor of ADAMTS-4/ADAMTS-5, and blocks both IL-1 induced aggrecan degradation in pig cartilage, as well as accelerated aggrecan breakdown in human OA cartilage.

1. Synthesis of Hydroxamic Acids

Hydroxamic acids were prepared as described in scheme 1 below.

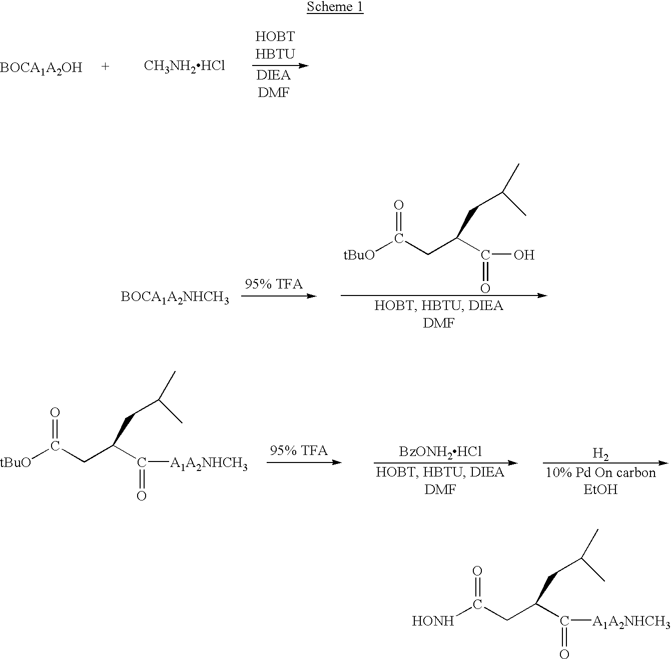

where A₁ and A₂ are the same or different amino acids.

In some cases A₂ need not be present.

Boc protected acids were coupled with methyl amine to form N-methyl amides. Boc groups were then removed and the TFA salts were coupled with mono succinic acids. The tert butyl groups were removed and the carboxylic acids were coupled with O-benzyl hydroxylamine to form protected hydroxamic acids. Hydrogenation over the Pd on carbon gave the corresponding hydroxamic acids.

2. Preparation of the Novel Inhibitors Comprising JWD40 and Synthetic Peptide Analogues Containing the GAG-Binding Sequence GGWGPWGPWG. (Seq. No. 3)

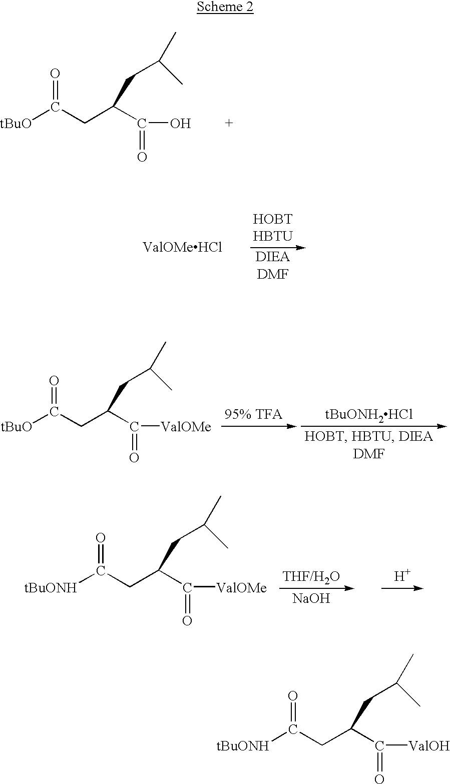

IMPORTANT—Conjugation of the hydroxamic acid analogs such as JWD40 to the synthetic peptide analogs can be accomplished through preparaton of intermediates similar to the one described above in Scheme 2. Monosuccinic acid was coupled with ValOMe.HCl to form the amide. Butyl group was removed. The formed acid was coupled with t-butyl protected hydroxyl amine salt to give protected hydroxamic acid. The methyl ester was hydrolized to give the corresponding acid which is conjugated to the synthetic peptide analogues using standard solid phase peptide conjugation techniques.

FIG. 5 illustrates the structure of one of the hydroxamic acid thrombospondin peptide inhibitors of aggrecanase.

Experimental

The following preparations and examples serve to illustrate the invention. They should not be construed as narrowing it, or limiting its scope in any way.

The starting material compounds, solvents, reagents, etc. described herein are available from commercial sources or are easily prepared from literature references by one of skill in the art. See Chem Sources USA, published annually by Directories Publications, Inc. of Boca Raton, Fla. Also see The Aldrich Chemical Company Catalogue, Milwaukee, Wis. The starting materials are used as obtained unless otherwise noted.

### EXAMPLE 1

### Preparation of 3-(t-Butyloxycarbonyl)-2′-(isobutylprpionylo-L-valine-O-methylester

Monosuccinic acid (3.0 g, 13.0 mmol), ValOMe.HCl (1.71 g, 13.0 mmol), HOBT (1.76 g, 13.0 mmol), HBTU (4.95 g, 13.0 mmol) were dissolved in DMF (20 ml). DIEA (6.8 ml, 39 mmol) was added. Stirred for 1 hr. EtOAc (100 ml) was added. Washed with water, sat NaHCO₃, NaCl. Dried over MgSO₄. Solvent was removed. The residue was purified on silica gel. (eluting with 50% EtOAc in hexanes). Yield: 3.5 g (87.5%). MS(EI): MH+=307. TLC:Rf (ethyl acetate:hexane=1:1)=0.56.

### EXAMPLE 2

### Preparation of 3-(t-Butyloxyaminocarbonyl)-2′-(isobutylpropionyl)-L-valine-O-methylester

3-(t-Butyloxycarbonyl)-2′-(isobutylpropionyl)-L-valine-O-methylester (2.0 g, 6.5 mmol) was dissolved in 95% TFA (20 ml). Stirred for 1 hr. Stripped down. Chased with hexanes. Pumped dry. To it in DMF (10 ml), were added tBuONH₂.HCl (819 mg, 6.5 mmol), HOBT (877 mg, 6.5 mmol), HBTU (2.46 g, 6.5 mmol), DIEA (3.4 ml, 6.5 mmol). Stirred for 1 hr. EtOAc (100 ml) was added. Washed with water, sat NaHCO3, NaCl. Dried over MgSO₄. Solvent was removed. The residue was purified on silica gel. (eluting with 50% EtOAc in hexanes). Yield: 1.5 g (72%). MS(EI): MH+=322. TLC:Rf (ethyl acetate:hexane=1:1)=0.45.

### EXAMPLE 3

### Preparation of 3-(t-Butyloxyaminocarbonyl)-2′-(isobutylpropionyl)-L-valine

3-(t-Butyloxyaminocarbonyl)-2′-(isobutylpropionyl)-L-valine-O-methylester (1.5 g, 4.67ml) was dissolved in methanol (20 ml), NaOH (1N, 6.5 ml) was added and the reaction was stirred for 1 hr at room temperature. The methanol was removed and water (20 ml) was added. The resulting water solution was washed with EtOAc (30 ml) and the aqueous layer was acidified with 1N Hcl and the resulting mixture extracted with EtOAc (2×30 ml). Dried over MgSO₄. Solvent was removed to give the corresponding acid. Yield: 1.2 g (83%). MS(EI): MH+=308.

### EXAMPLE 4

### General Procedure A: Preparation of BOC Amino Acid N-methyl Amides

A BOC-protected amino acid (1.0 eq) was dissolved in CH₂Cl₂. CDI (1.33 eq) was added. After stirring for 0.5 hr, methylamine.HCl (1.33 eq) was added followed by triethyl amine (1.33 eq). Stirred for 1 hr at 0° C., and overnight at RT. Washed with 1 N HCl, sat NaHCO₃, NaCl. Dried over MgSO₄, filtered and concentrated to the amide.

### EXAMPLE 5

### General Procedure B: Preparation of Amino Acid N-methyl Amides—Succinic Acid Adducts

BOC Amino acid N-methyl amide (1 eq) was dissolved in 95% TFA. Stirred for 1 hr. Stripped down. Chased with hexanes. Pumped dry. To it in DMF, were added a substituted succinic acid mono t-butylester (prepared according to the published procedure, see reference) (1 eq), HOBT (1 eq), HBTU (1 eq), DIEA (3 eq). Stirred for 1 hr. EtOAc was added. Washed with water, sat NaHCO₃, NaCl. Dried over MgSO₄. Solvent was removed. The residue was purified on silica gel.. (50% EtOAc in hexanes).

### EXAMPLE 6

### General Procedure C: Preparation of Hydroxamic Acids

Amino acid N-methyl amides—Succinic Acid Adducts (1 eq) was dissolved in 95% TFA. Stirred for 1 hr. Stripped down. Chased with hexanes. Pumped dry. To it in DMF, were added BzONH₂. HCl. (1 eq), HOBT (1 eq), HBTU (1 eq), DIEA (3 eq). Stirred for 1 hr. EtOAc was added. Washed with water, sat NaHCO₃, NaCl. Dried over MgSO₄. Solvent was removed. The residue was purified on silica gel. (50% EtOAc in hexanes).

Benzyl protected hydroxamic acids (1 eq) were dissolved in MeOH. 10% Pd on carbon (10% of the weight of Benzyl protected hydroxamic acids) was added. Hydrogenated with H₂ (100 Psi) for 3 hr. Filtered through CELITE. Concentrated to a crude solid. Recrystallized with EtOAc to give the pure hydroxamic acids.

### EXAMPLE 7

### N-BOC-L-valine-N-methylamide

This compound was prepared according to general procedure A. Yield=90%. MS(EI): MH⁺=231. TLC: Rf (ethyl acetate: hexane=1:1)=0.42.

### EXAMPLE 8

### N-BOC-L-leucine-N-methylamide

This compound was prepared according to general procedure A. Yield=62%. MS(EI): MH⁺=244. TLC: Rf (ethyl acetate: hexane=1:1)=0.40.

### EXAMPLE 9

### N-BOC-L-phenylalanine-N-methylamide

This compound was prepared according to general procedure A. Yield=95%. MS(EI): MH⁺=279. TLC: Rf (ethyl acetate: hexane=1:1)=0.46.

### EXAMPLE 10

### N-BOC-L-homophenylalanine-N-methylamide

This compound was prepared according to general procedure A. Yield=95%. MS(EI): MH⁺=293. TLC: Rf (ethyl acetate: hexane=1:1)=0.45.

### EXAMPLE 11

### N-BOC-L-tyrosine(bzl)-N-methylamide

This compound was prepared according to general procedure A. Yield=97%. MS(EI): MH⁺=385. TLC: Rf (ethyl acetate: hexane=1:1)=0.32.

### EXAMPLE 12

### N-BOC-L-4-fluorophenylalanine-N-methylamide

This compound was prepared according to general procedure A. Yield=90%. MS(EI): MH⁺=297. TLC: Rf (ethyl acetate: hexane=1:1)=0.43.

### EXAMPLE 13

### N-BOC-L-alanine-N-methylamide

This compound was prepared according to general procedure A. Yield=38%. MS(EI): MH⁺=203. TLC: Rf (ethyl acetate: hexane=1:1)=0.42.

### EXAMPLE 14

### 3-(t-Butyloxycarbonyl)-2(R)-(isobutylpropionyl)-L-valine-N-methylamide

This compound was prepared according to general procedure B. Yield=73%. MS(EI): MH⁺=343. TLC: Rf (ethyl acetate: hexane=1:1)=0.31.

### EXAMPLE 15

### 3-(t-Butyloxycarbonyl)-2(R)-(isobutylpropionyl)-L-leucine-N-methylamide

This compound was prepared according to general procedure B. Yield=94%. MS(EI): MH⁺=356. TLC: Rf (ethyl acetate: hexane=1:1)=0.30.

### EXAMPLE 16

### 3-(t-Butyloxycarbonyl)-2(R)-(isobutylpropionyl)-L-phenylalanine-N-methylamide

This compound was prepared according to general procedure B. Yield=64%. MS(EI): MH⁺=390. TLC: Rf (ethyl acetate: hexane=1:1)=0.30.

### EXAMPLE 17

### 3-(t-Butyloxycarbonyl)-2(R)-(isobutylpropionyl)-L-tyrosine(bzl)-N-methylamide

This compound was prepared according to general procedure B. Yield=60%. MS(EI): MH⁺=497. TLC: Rf (ethyl acetate: hexane=1:1)=0.29.

### EXAMPLE 18

### 3-(t-Butyloxycarbonyl)-2(R)-(isobutylpropionyl)-L-4-fluorophenylalanine-N-methylamide

This compound was prepared according to general procedure B. Yield=73%. MS(EI): MH⁺=409. TLC: Rf (ethyl acetate: hexane=1:1)=0.31.

### EXAMPLE 19

### 3-(t-Butyloxycarbonyl)-2(R)-(isobutylpropionyl)-L-alanine-N-methylamide

This compound was prepared according to general procedure B. Yield=50%. MS(EI): MH⁺=315. TLC: Rf (ethyl acetate: hexane=1:1)=0.31.

### EXAMPLE 20

### 3-(t-Hydroxycarbamoyl)-2(R)-(isobutylpropionyl)-L-valine-N-methylamide

This compound was prepared according to general procedure C. Yield=58%. MS(EI): MH⁺=302. TLC: Rf (MeOH; CH₂Cl₂=1:9)=0.54.

### EXAMPLE 21

### 3-(t-Hydroxycarbamoyl)-2(R)-(isobutylpropionyl)-L-leucine-N-methylamide

This compound was prepared according to general procedure C. Yield=32%. MS(EI): MH⁺=315. TLC: Rf (MeOH; CH₂Cl₂=1:9)=0.55.

### EXAMPLE 22

### 3-(t-Hydroxycarbamoyl)-2(R)-(isobutylpropionyl)-L-phenylalanine-N-methylamide

This compound was prepared according to general procedure C. Yield=46%. MS(EI): MH⁺=350. TLC: Rf (MeOH; CH₂Cl₂=1:9)=0.55.

### EXAMPLE 23

### 3-(t-Hydroxycarbamoyl)-2(R)-(isobutylpropionyl)-L-homophenylalanine-N-methylamide

This compound was prepared according to general procedure C. Yield=62%. MS(EI): MH⁺=364. TLC: Rf (MeOH; CH₂Cl₂=1:9)=0.55.

### EXAMPLE 24

### 3 -(t-Hydroxycarbamoyl)-2(R)-(isobutylpropionyl)-L-tyrosine(bzl)-N-methylamide

This compound was prepared according to general procedure C. Yield=37%. MS(EI): MH⁺=456. TLC: Rf (MeOH; CH₂Cl₂=1:9)=0.54.

### EXAMPLE 25

### 3 -(t-Hydroxycarbamoyl)-2(R)-(isobutylpropionyl)-L-4-fluorophenylalanine-N-methylamide

This compound was prepared according to general procedure C. Yield=45%. MS(EI): MH⁺=368. TLC: Rf (MeOH; CH₂Cl₂=1:9)=0.55.

### EXAMPLE 26

### 3-(t-Hydroxycarbamoyl)-2(R)-(isobutylpropionyl)-L-alanine-N-methylamide

This compound was prepared according to general procedure C. Yield=67%. MS(EI): MH⁺=274. TLC: Rf (MeOH; CH₂Cl₂=1:9)=0.54.

### EXAMPLE 27

### Inhibitor Activity Measured in Purified Bovine Aggrecan Matrices

Digestions were carried out in 100 μl of 50 mM Tris/HCl buffer, pH 7.5, containing 100 mM NaCl and 10 mM CaCl₂. Human recombinant ADAMTS-4/ADAMTS-5 were prepared as described (1, 2). Purified bovine aggrecan (12) (500 nM) was incubated with 5 nM ADAMTS-4/ADAMTS-5 at 37° C. for 2 hours, in the absence or presence of each of the hydroxamates described above, at concentrations ranging from 0.1 to 100 nM. Following the incubation, cleavage of aggrecan at the Glu¹⁴⁸⁰-¹⁴⁸¹Gly bond was monitored by Western Blot analysis, using the neoepitope antibody that recognizes the new C-terminus GELE¹⁴⁸⁰, as previously described (12).

### EXAMPLE 28

### Inhibitor Activity Measured in Pig Articular Cartilage Cultures

Articular cartilage was dissected from the knees of young pigs. Cartilage was allowed to equilibrate for 3 days in DMEM supplemented with 10% FCS, penicillin (100 U/ml) and streptomycin (100 μg/ml). Subsequently, cartilage was cut into 3×3 mm explants, weighing approximately 10-20 mg each, and incubated in 96-well plates for 72 hours with either control medium (serum-free DMEM),ml), IL-1α (100 ng/ml), or IL-1α (100 ng/ml) plus a series of hydroxamic acids at a concentration range of 0.1 μM to 10 μM. At the end of the culture period, glycosaminoglycan (GAG) levels in the culture media were determined by dimethylmethylene blue (DMMB) assay, as described by Farndale et al (13).

### EXAMPLE 29

### Hydroxamic Acid Thrombospondin Peptide Analog that Inhibits Aggreanase Activity

Articular cartilage was dissected from the hips of patients with osteoarthritis at the time of joint replacement. Cartilage was allowed to equilibrate for 3 days in DMEM supplemented with penicillin (100 U/ml) and streptomycin (100 μg/ml). Subsequently, cartilage was cut into 3×3 mm explants, weighing approximately 10-20 mg each, and incubated in 96-well plates for 48 hours in the presence or absence of JWD40 at a concentration range of 0.1 μM to 10 μM. At the end of the culture period, the media were analyzed for aggrecan fragments generated by cleavage of aggrecan by aggrecanase, using a neoepitope that recognizes the new N-terminus ¹⁴⁸⁰ARGS (14).

### EXAMPLE 30

### Inhibition of ADAMTS-4 Cleavage of Aggrecan by Peptide Inhibitors

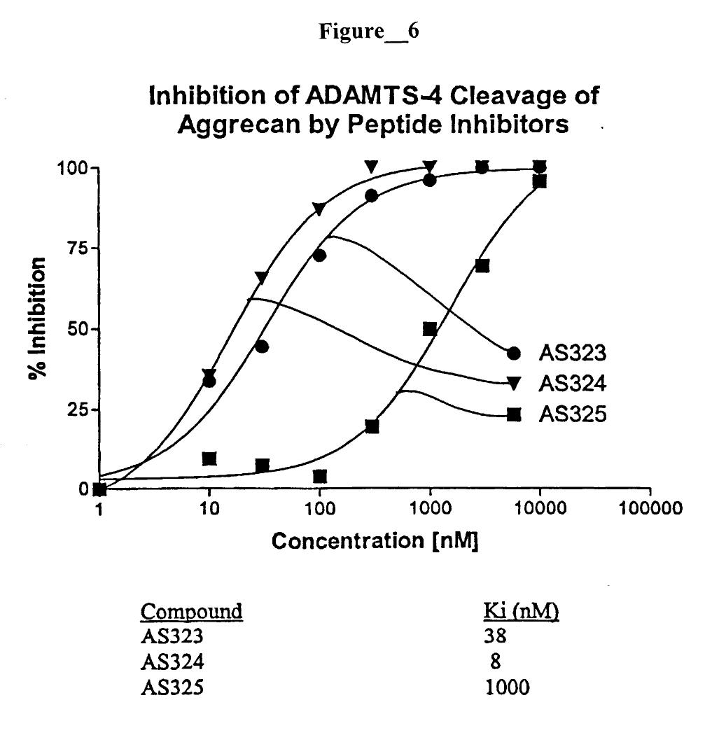

Several different thrombospondin peptides with varying spacer lengths hydroxamic were prepared. The resulting compounds were analyzed for their ability to inhibit ADAMTS4 (aggrecanase 1). See FIG. 6. In the analysis, 25 pmolar of ADAMTS4 was incubated with 500 nmolar of bovine aggrecan monomer for 4 hrs in the abscence or presence of each of the hydroxamic acid thrombospondin peptides at various concentrations ranging from 1000 to 1 nmolar. Following the incubation the reactions were quenched with 50 mmolar EDTA and the aggrecan products analyzed by ELISA for fragments containing the amino acid sequence ³⁷³ARGS that results when ADAMTS4 cleaves bovine aggrecan at residues Glu³⁷³/Ala³⁷⁴. The data in FIG. 6 show that the hydroxamic acid linked to the thrombospondin peptide SNISQAGGWGPWGPWGDSSAT (AS324) (Seq. No. 10) with 6 amino acids (11 Å) separating the GAG binding motif and the hydroxamic acid had the best potency with a Ki value of 8.8 nmolar. If the spacer between the GAG binding motif and the hydroxamic acid increased to 12 residues (33Å) as in the case of peptide MDQLQDSNISQAGGWGPWGPWGDSSAT (AS325) (Seq. No. 12), compound potency decreased with a Ki value of 1064 nmolar. Thus, this demonstrates that the number of amino acids separating the GAG binding portion of the peptide and the hydroxamic acid is critical for compound potency for inhibition.

Table 1 compares the potency and selectivity of the JW compounds.

TABLE 1

Potency and Selectivity of JW Compounds
Apparent Ki Values [nM]*

| | Collagenase | gelatinase | stromelysin | aggrecanase | Articular* |
|---|---|---|---|---|---|
| Compound | MMP-1 | MMP-2 | MMP-3 | ADAM-TS4/5 | Cartilage |
| JWC-95 | 5.9 | 2.3 | 65 | 98 | 2900 |
| JWC-96 | 5.6 | 2.9 | 102 | 38 | 1100 |
| JWC-97 | 7.4 | 2.3 | 135 | 62 | 1800 |
| JWC-100 | 1.0 | <0.1 | 0.9 | 160 | 6000 |
| JWD-18 | 995 | >1000 | >1000 | >10,000 | >100,000 |
| JWD-39 | 22 | 27 | 73 | 38 | 1888 |
| JWD-40 | 26 | 36 | 125 | 17 | 2048 |
| JWD-52 | 76 | 59 | 1000 | 35 | — |
| XN908 | 3.8 | 2.7 | 5 | 53 | 6300 |
| XS309 | 0.8 | 14 | 12 | >10,000 | >10,000 |

*Ki = IC50/(1 + [S]/Km)

### Utility and Administration

The compounds of the invention have been shown to inhibit aggrecanase in various in vivo and in vitro animal preparations and tissue cultures, and accordingly are useful in the affecting physiological phenomena. These compounds have been shown to be effective in animal models and are, therefore, useful in treating a mammal, particularly a human being.

These compounds are useful as immunosuppressants, and in particular they are useful in the treatment of autoimmune diseases, such as arthritis, etc.

Administration of the active compounds and salts described herein can be via any of the accepted modes for administration for therapeutic agents which inhibit aggrecanase. These methods include oral, parenteral, transdermal, subcutaneous and other systemic modes. The preferred method of administration is oral, except in those cases where the subject is unable to ingest, by himself, any medication. In those instances it may be necessary to administer the composition parenterally.

Depending on the intended mode, the compositions may be in the form of solid, semi-solid or liquid dosage forms, such as, for example, tablets, suppositories, pills, capsules, powders, liquids, suspensions, skin patch, or the like, preferably in unit dosage forms suitable for single administration of precise dosages. The compositions will include a conventional pharmaceutical excipient and an active compound of formula I or the pharmaceutically acceptable salts thereof and, in addition, may include other medicinal agents, pharmaceutical agents, carriers, adjuvants, diluents, etc.

The amount of active compound administered will, of course, be dependent on the subject being treated, the severity of the affliction, the manner of administration and the judgement of the prescribing physician. However, an effective dosage is in the range of 0.1-100 mg/kg/day, preferably 0.5-5 mg/kg/day. For an average 70 kg human, this would amount to 7-7000 mg per day, or preferably 35-350 mg/day. Alternatively, the administration of compounds as described by L. C. Fritz et al. in U.S. Pat. No. 6,200,969 is followed. One of skill in the art with this disclosure can create an effective pharmaceutical formulation.

Since the effects of the compounds herein are achieved through the same central mechanism (inhibition of aggrecanase in the living system) dosages (and forms of administration) are within the same general and preferred ranges for all these utilities.

For solid compositions, conventional non-toxic solid include, for example, pharmaceutical grades of mannitol, lactose, starch, magnesium stearate, sodium saccharin, talcum, cellulose, glucose, sucrose, magnesium carbonate, and the like may be used. The active compound as defined above may be formulated as suppositories using, for example, polyalkylene glycols (e.g. propylene glycol) as the carrier. Liquid pharmaceutically administrable compositions can, for example, be prepared by dissolving, dispersing, etc. an active compound as defined above and optional pharmaceutical adjuvants in a excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington 's Pharmaceutical Sciences, Mack Publishing Company, Easton, Pa., 17^{th} Edition, 1985. The composition or formulation to be administered will, in any event, contain a quantity of the active compound(s), a therapeutically effective amount, i.e. in an amount effective to alleviate the symptoms of the subject being treated.

For oral administration, a pharmaceutically acceptable non-toxic composition is formed by the incorporation of any of the normally employed excipients, such as, for example pharmaceutical grades of mannnitol, lactose, starch, magnesium stearate, sodium saccharin, talcum, cellulose, glucose, sucrose, magnesium, carbonate, and the like. Such compositions take the form of solutions, suspensions, tablets, pills, capsules, powders, sustained release formulations and the like. Such compositions may contain 10%-95% active ingredient, preferably 1-70%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, etc. Injection is a preferred mode.

A recent approach for parenteral administration employs the implantation or skin patch for a slow-release or sustained-release system, such that a constant level of dosage is maintained. See. e.g., U.S. Pat. No. 3,710,795, which is incorporated herein by reference.

The following preparations and examples serve to illustrate the invention. They should not be construed as narrowing it, or limiting its scope in any way.

### Conclusions

The design of these novel hydroxamic acid thrombospondin peptide analog inhibitors for:

a) allows the compound to localize and accumulate in the cartilage extracellular matrix, specifically bound to aggrecan,

b) allows for the hydroxamic acid to be in position to effectively inhibit the cartilage aggrecanases, ADAMTS-4/ADAMTS-5, and

c) allows for the treatment of diseases characterized by cartilage degradation, such as osteoarthritis, rheumatoid arthritis, spondylarthropathies, septic arthritis, and other diseases characterized by cartilage degradation.

d) the hydroxamic acids of FIGS. 1A to 1J when bonded with 1 to 5, 1 to 10, 1 to 20, 1 to 30 or more amino acids or 5 to 30 or more amino acids are useful pharmaceutical agents for the diseases of c).

While only a few embodiments of the invention have been shown and described herein, it will become apparent to those skilled in the art that various modifications and changes can be made in the hydroxamic acid analogs as aggrecanase inhibitors, their synthesis and their pharmaceutical uses without departing from the spirit and scope of the present invention. All such modifications and changes coming within the scope of the appended claims are intended to be carried or thereby.

SEOUENCE DATA
Antagonist sequence:

| | | | |
|---|---|---|---|
| CMGGRCLHMD | (Seq. No.1) | QLQDFNIPQA | (Seq. No.2) |
| GGWGPWGPWG | (Seq. No.3) | DCSRTCGGGV | (Seq. No.4) |

Mutations

| | | | |
|---|---|---|---|
| SMGGRSLHMD | (Seq. No.5) | QLQDSNISQA | (Seq. No.6) |
| GGWGPWGPWG | (Seq. No.7) | DSSAT | (Seq. No.8) |

Hydroxaminic Acid Peptides Analogs synthesized:

| | |
|---|---|
| For the hydroxamic acid structure: | HO—NH—(C═O)—CH—CH(CH(CH₃))—(C═O)— |

| | | |
|---|---|---|
| Hydroxamic Acid-V-SQAGGWGPWGPWGDSSAT | (˜11′A) | (Seq. No.9) |
| Hydroxamic Acid-V-SNISQAGGWGPWGPWGDSSAT | (˜22′A) | (Seq. No.10) |
| Hydroxamic Acid-V-LQDSNISQAGGWGPWGPWGDSSAT | (˜33′A) | (Seq. No.11) |
| Hydroxamic Acid-V-MDQLQDSNISQAGGWGPWGPWGDSSAT | (˜44′A) | (Seq. No.12) |

This can be done in one synthesis by removing 25% of the resin after
cycle 18, 21, and 24. The hydroxamate analog would then be attached
through the amino terminus of the peptide.

| | | | | |
|---|---|---|---|---|
| SIGNAL | 1 | 51 | | POTENTIAL |
| FT PROPEP | | 52 | 212 | |
| FT CHAIN | | 213 | 837 | ADAM-TS 4. |
| FT SITE | | 194 | 194 | CYSTEINE SWITCH (POTENTIAL). |
| FT METAL | | 361 | 361 | ZINC (CATALYTIC) (BY SIMILARITY). |
| FT ACT_SITE | | 362 | 362 | BY SIMILARITY. |
| FT METAL | | 365 | 365 | ZINC (CATALYTIC) (BY SIMILARITY). |
| FT METAL | | 371 | 371 | ZINC (CATALYTIC) (BY SIMILARITY). |
| FT DOMAIN | | 437 | 519 | DISINTEGRIN-LIKE |
| FT DOMAIN | | 520 | 576 | TSP-TYPE 1 1. |
| FT DOMAIN | | 577 | 685 | CYS-RICH. |
| FT DOMAIN | | 686 | 837 | SPACER. |
| FT DOMAIN | | 247 | 252 | POLY-ALA. |
| FT CARBOHYD | | 68 | 68 | N-LINKED (CLCNAC...) (POTENTIAL). |
| FT CONFLICT | | 77 | 77 | A->T (IN REF. 1). |

| | | |
|---|---|---|
| SQ SEQUENCE | 837 AA; 90224 MW; 5DF9C9AC137DF41FCRC64; | (Seq. No.13.) |

MSQTGSHPGR GLAGRWLWGA QPCLLLPIVP LSWLVWLLLL LLASLLPSAR LASPLPREEE
IVFPEKLNGS VLPGSGAPAR LLCRLQAFGE TLLLELEQDS GVQVEGLTVQ YLGQAPELLG
GAEPGTYLTG TINGDPESVA SLHWDGGALL GVLQYRGAEL HLQPLEGGTP NSAGGPGAHI
LRRKSPASGQ GPMCNVKAPL GSPSPRPRRA KRFASLSRFV ETLVVADDKM AAFHGAGLKR
YLLTVMAAAA KAFKHPSIRN PVSLVVTRLV ILGSGEEGPQ VGPSAAQTLR SPCAWQRGLN
TPEDSDPDHF DTAILPTRQD LCGVSTCDTL GMADVGTVCD PARSCAIVED DGLQSAFTAA
HELGHVFNML HDNSKPCISL NGPLSTSRHV MAPVMAHVDP EEPWSPCSAR FITDFLDNGY
GHCLLDKPEA PLHLPVTFPG KDYDADRQCQ LTPGPDSRHC PQLPPPCAAL WCSGHLNGHA
MCQTKHSPWA DGTPCGPAQA CMGGRCLHMD QLQDFNIPQA GGWGPWGPWG DCSRTCGGGV
QFSSRDCTRP VPRNGGKYCE GRRTRFRSCN TEDCPTGSAL TFREEQCAAY NHRTDLFKSF
PGPMDWVPRY TGVAPQDQCK LTCQARALGY YYVLEPRVVD GTPCSPDSSS VCVQGRCIHA
GCDRIIGSKK KFDKCMVCGG DGSGCSKQSG SFRKFRYGYN NVVTIPAGAT GILVRQQGNP
GHRSIYLALK LPDGSYALNG EYTLMPSPTD VVLPGAVSLR YSGATAASET LSGHGPLAQP
LTLQVLVAGN PQDTRLRYSF FVPRPTPSTP RPTPQDWLHR RAQILEILRR RPWAGRK

| | |
|---|---|
| HO—NH—(C═O)—CH₂—CH(—CH₂—CH(CH₃)₂)—(C═O)—V--QAGGWGPWGPWGDSSAT | (˜11′A); |
| HO—NH—(C═O)—CH₂—CH(CH(CH₃)₂)—(C═O)—V--SNISQAGGWGPWGPWGDSSAT | (˜22′A); |
| HO—NH—(C═O)—CH₂—CH(CH(CH₃)₂)—(C═O)—V--LQDSNISQAGGWGPWGPWGDSSAT | (˜33′A); and |
| HO—NH—(C═O)—CH₂—CH(CH(CH₃)₂)—(C═O)—V--MDQLQDSNISQAGGWGPWGPWGDSSAT | (˜44′A) |

We claim:

1. The therapeutic use of hydroxamic acid thrombospondin peptide analog compounds for the treatment of diseases characterized by in vivo cartilage degradation, selected from osteoarthritis, rheumatoid arthritis, spondylarthropathies, and septic arthritis.

2. The use of hydroxamic acid thrombospondin peptide analog compounds for the inhibition of ADAMTS-4/ADAMTS-5 in vitro or in vivo.

3. The use of the compounds of claim 1 as a system for the delivery of small molecule enzyme inhibitors into the tissue through specific interaction with the endogenous substrate.

4. The hydroxamic acids having the structures suitable for use in preparing thrombospondin peptide analog compounds found in FIGS. 1A to 1J as shown: 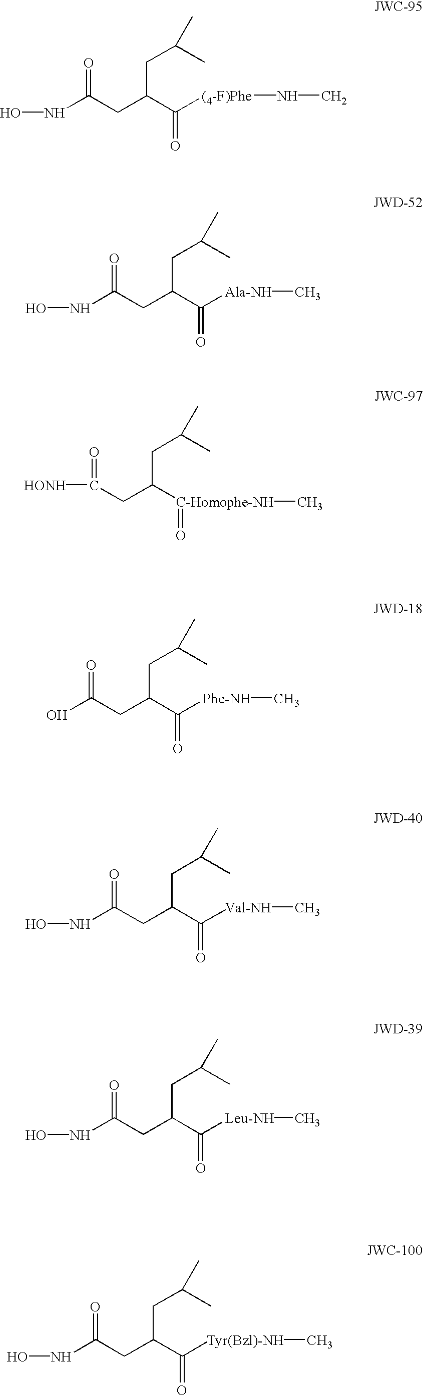
wherein for the hydroxamic acid structure HO—NH—(C═O)—CH₂—CH(—CH₂—CH(CH₃)₂)—(C═O)—V—, the V (valine) can be another amino acid to connect to the amino acid sequence of about 10 or more amino acids.

5. The hydroxamic acid structure of claim 4 where the V (valine) is replaced by an amino acid selected from the group consisting of alanine, arginine, asparagine, cysteine, glutamine, glycine, histidine, isoleucine, lysine, methionine, phenylalanine, proline, serine, threonine, tryptophan, and tyrosine.

6. The compounds selected from the group:

| | |
|---|---|
| HO—NH—(CO═O)—CH₂—CH(—CH₂—CH(CH₃)₂)—(C═O)—V— | |
| —QAGGWGPWGPWGDSSAT; | (˜11′A) |
| HO—NH—(C═O)—CH₂—CH(CH(CH₃)₂)—(C═O)—V— | |
| SNISQAGGWGPWGPWGDSSAT; | (˜22′A); |
| HO—NH—(C═O)—CH₂—CH(CH(CH₃)₂—(C═O)—V— | |
| —LQDSNISQAGGWGPWGPWGDSSAT; and | (˜33′A) |
| HO—NH—(C═O)—CH₂—CH(CH(CH₃)₂)—(C═O)—V— | |
| MDQLQDSNISQAGGWGPWGPWGDSSAT | (˜44′A) |

7. A method of treatment to arrest cartilage degradation in vivo in a mammal preferably a human, which method comprises administering a therapeutically effective amount of a compound of claim 4 to a subject in need of treatment.

8. A method of treatment to arrest cartilage degradation in vivo in a mammal preferably a human, which method comprises administering a therapeutically effective

9. The compound of claim 6 which is

| | |
|---|---|
| HO—NH—(C═O)—CH₂—CH(—CH₂—CH(CH₃)₂)—(C═O)—V- | (˜11′A) |
| -QAGGWGPWGPWGDSSAT | |

10. The compound of claim 6 which is

| | |
|---|---|
| HO—NH—(C═O)—CH₂—CH(CH(CH₃)₂)—(C═O)—V--SNIS | (˜22′A) |
| QAGGWGPWGPWGDSSAT | |

11. A pharmaceutical compositions for use in the treatment of diseases characterized by in vivo cartilage degration, which composition comprises structures selected from the groups consisting of 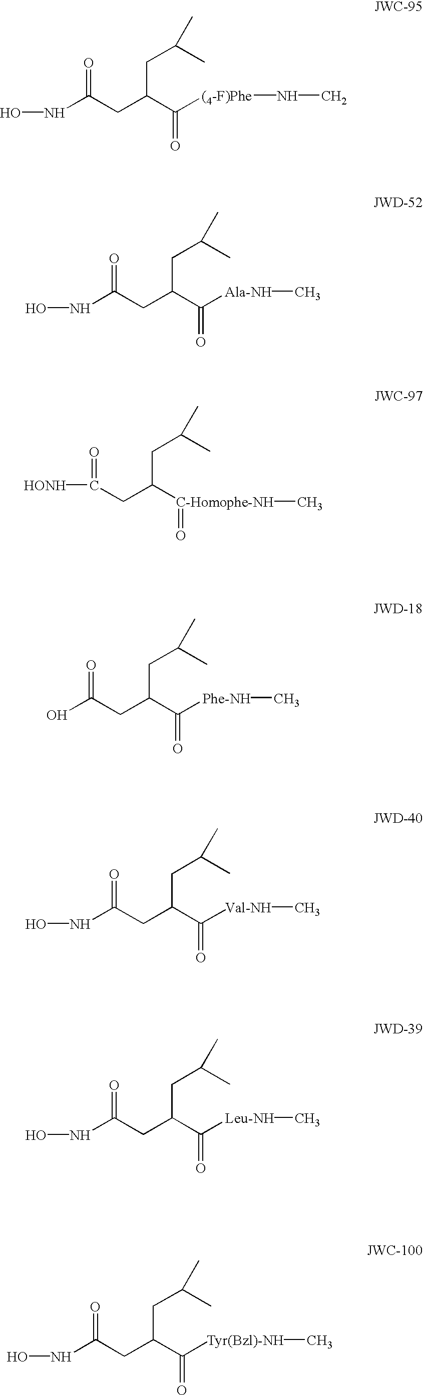
characterized by in vivo cartilage degration, which composition compresses structures selected from the group consisting of

| | |
|---|---|
| HO—NH—(C═O)—CH₂—CH(—CH₂—CH(CH₃)₂)—(C0)—V--QAGGWGPWGPWGDSSAT | (˜11′A); |
| HO—NH—(C═O)—CH₂—CH(CH(CH₃)₂)—(C═O)—V--SNISQAGGWGPWGPWGDSSAT | (˜22′A); |
| HO—NH—(C═O)—CH₂—CH(CH(CH₃)₂)—(C═O)—V--LQDSNISQAGGWGPWGPWGDSSAT | (˜33′A); and |
| HO—NH—(C═O)—CH₂—CH(CH(CH₃)₂)—(C═O)—V--MDQLQDSNISQAGGWGPWGPWGDSSAT | (˜44′A) |

and a pharmaceutically acceptable excipient.

13. The pharmaceutical composition of claim 12 as

| | |
|---|---|
| HO—NH—(C═O)—CH₂—CH(—CH₂—CH(CH₃)₂)—(C═O)—V- | (˜11′A) |
| -QAGGWGPWGPWGDSSAT | |

14. The pharmaceutical composition of claim 12 as

| | |
|---|---|
| HO—NH—(C═O)—CH₂—CH(CH(CH₃)₂)—(C═O)—V--SNIS | (˜22′A) |
| QAGGWGPWGPWGDSSAT | |

15. The pharmaceutical composition of claim 12 as

| | |
|---|---|
| HO—NH—(C═O)—CH₂—CH(CH(CH₃)₂)—(C═O)—V--LQDS | (˜33′A) |
| NISQAGGWGPWGPWGDSSAT | |

16. The pharmaceutical composition of claim 11 wherein said groups are covalently bonded to amino acid chains having 5 to 30 amino acids.
